(19) United States
(12) Patent Application Publication
(10) Pub. No.: US 2009/0186009 A1
(43) Pub. Date: Jul. 23, 2009

(54) SOLID MATTER CONTAINING COENZYME Q

(75) Inventors: Masao Sato, Hyogo (JP); Takashi Ueda, Hyogo (JP); Yoshiyuki Shinagawa, Hyogo (JP); Shinsuke Akao, Hyogo (JP); Naohiro Imai, Hyogo (JP); Shiro Kitamura, Hyogo (JP)

Correspondence Address: SUGHRUE MION, PLLC, 2100 PENNSYLVANIA AVENUE, N.W., SUITE 800, WASHINGTON, DC, 20037, US

(73) Assignee: Kaneka Corporation, Osaka-shi, Osaka (JP)

(21) Appl. No.: 12/298,449

(22) PCT Filed: Apr. 24, 2007
(86) PCT No.: PCT/JP2007/058844

§ 371 Date: Feb. 9, 2009

(30) Foreign Application Priority Data
Apr. 24, 2006 (JP) 2006-119409

(51) Int. Cl. A61K 31/122

(52) U.S. Cl. 424/94.1

(57) ABSTRACT

The present invention provides a coenzyme Q-containing solid wherein coenzyme Q fine particles are dispersed in particles made of a physically crosslinked polymer. In addition, the present invention provides a method of producing a coenzyme Q-containing solid, which includes a step of bringing an atomized water-soluble polymer solution capable of forming a physical gel, wherein coenzyme Q is dispersed, into contact with an atomized gelling solution.

In the solid of the present invention, the disintegratability is suppressed in the stomach, and rapidly disintegrated in the intestine. Accordingly, coenzyme Q is more quickly absorbed from the intestine, which in turn enhances the absorbability of coenzyme Q from the intestine. In addition, the production method of the present invention is industrially advantageous.

### TECHNICAL FIELD

The present invention relates to a solid such as granule etc. comprising coenzyme Q such as ubiquinone and the like, and a production method thereof.

### BACKGROUND ART

Coenzyme Q is known to include coenzymes Q1 to Q13 depending on the number of repeat structures in the isoprene side chain, and coenzyme Q10 is mainly applied to mammals. Coenzyme Q, particularly coenzyme Q10, is localized in mitochondria, lysosome, golgi, microsome, peroxisome, cellular membrane and the like, and in mitochondria, it is a substance essential for the functional maintenance of living organisms and known to be involved in the activation of ATP production, antioxidant action in vivo and membrane stabilization, as a constituent component of the electron transport system. Coenzyme Q is known to include an oxidized form and a reduced form.

Oxidized coenzyme Q is also named as ubiquinone, which is a quinone compound widely distributed in the living organisms. Of ubiquinones, ubidecarenone present in higher animals including human is known as a substance having a vitamin-like function, which has not only a biological activity as coenzyme but also a function to improve enzyme utilization efficiency. Ubidecarenone is oxidized coenzyme Q10, which is produced as a metabolic cardiant in the form of a pharmaceutical product. In recent years, moreover, utilization thereof for food has become available after revision of the food-drug classification in Japan, and ubidecarenone is now applied to the field of health foods. Oxidized coenzyme Q10 is considered to be an essential coenzyme for the production of adenosine triphosphate in mitochondria, and reportedly provides a cardiac muscle protecting action, an antiaging action, a cardiac function improvement, a blood pressure increase suppressive action and the like, resulting from improved immune function.

As coenzyme Q, moreover, there are known not only oxidized type but also reduced coenzyme Q, ubiquinol. Since the antioxidant activity is exclusively shown by ubiquinol, and coenzyme Q in the body mostly exists as ubiquinol, ubiquinol is considered the main form. However, since ubiquinol lacks oxidization stability, ubiquinone is often used industrially.

Nevertheless, coenzyme Q such as ubiquinone and the like is a slightly water-soluble substance, whose dissolution rate in the gastrointestinal tract is slow. Accordingly, its absorption in the body is slow and its bioavailability is low unless treated some way.

In view of the above, studies have been made to improve the absorbability. Focusing on ubiquinone, patent reference 1 discloses a preparation comprising ubiquinone included in cyclodextrin, and patent reference 2 discloses a liposome preparation containing ubiquinone. However, these methods have low practicality due to the production cost and complicated preparation steps. In addition, studies have been made to give an emulsion powder of ubiquinone. For example, in patent reference 3, ubiquinone is dispersed in or emulsified with an aqueous solution of an organic acid and a water-soluble substance such as gum arabic to form protective colloid, which is then added with an excipient and spray dried in a fluidized bed to give a preparation containing ubiquinone. However, the production method by spray drying requires cumbersome maintenance operation of the manufacturing machine such as cleaning of the preparation attached to the machine, and therefore, the development of a technique superior in the production cost and plant cost is currently desired.

On the other hand, studies have been conducted which aims at efficient absorption of a slightly water-soluble pharmaceutical agent in the body by including the agent in a safe polymer usable for pharmaceutical agents and foods. Alkali metal salt of alginic acid such as sodium alginate forms what is called Egg Box junction surrounding a polyvalent metal salt upon contact with the polyvalent (divalent or above) metal salt, and changes from a viscose fluid (zol) to an elastic solid (gel). Many studies have been made to include a slightly water-soluble drug in the alginate gel. According to non-patent reference 1, calcium alginate gel is known to disintegrate depending on pH, and therefore, a preparation that releases a liposoluble substance, inclusive of drugs, rapidly and efficiently in the intestine has been developed. In patent reference 4, for example, a preparation is disclosed which is produced by adding dropwise a suspension of a basic drug dispersed in a sodium alginate solution to a calcium chloride solution from a nozzle, and drying the alginate beads thus formed. However, the production method requires immersion in a calcium chloride solution for about 72 hr to allow gellation. Thus, the method is time-consuming and problematic in terms of operation efficiency.

patent reference 1: JP-A-60-89442
patent reference 2: JP-A-60-1124
patent reference 3: JP-B-3549197
patent reference 4: JP-A-2-167220
non-patent reference 1: Chem. Pharm. Bull 35 (4) 1555-1563 (1987)

### DISCLOSURE OF THE INVENTION

### Problems to be Solved by the Invention

To accelerate the intracorporeal absorption rate of slightly water-soluble coenzyme Q from the intestine, and further, to increase the amount of coenzyme Q absorbed intracorporeally, disintegration of the preparation itself needs to be suppressed in the stomach and become rapid in the intestine. This is probably because suppressed disintegration in the stomach and rapid disintegratability in the intestine inevitably increases the release rate and absorption rate of coenzyme Q in the intestine, which in turn increases the amount of coenzyme Q absorbed intracorporeally.

In view of the above-mentioned situation, the present invention aims to provide a solid containing coenzyme Q such as ubiquinone and the like, wherein the disintegratability of coenzyme Q in the stomach is suppressed and the disintegration in the intestine is rapid, and therefore, the absorbability of coenzyme Q in the body is high, as well as an industrially advantageous production method thereof.

### Means of Solving the Problems

The present inventors have conducted intensive studies in an attempt to solve the aforementioned problems and found that a solid comprising coenzyme Q dispersed as fine particles in a polymer can be obtained by bringing an atomized aqueous polymer solution comprising coenzyme Q into contact with an atomized gelling agent (coagulating agent), and the solid comprising coenzyme Q shows suppressed disintegratability in the stomach and is quickly disintegrated in the intestine, which resulted in the completion of the present invention.

Accordingly, the present invention relates to the following (1) to (16).

(1) A solid comprising coenzyme Q, wherein fine particles of coenzyme Q are dispersed in particles comprised of physically crosslinked polymers.
(2) The solid of (1), wherein the solid is a granule.
(3) The solid of (1) or (2), wherein the coenzyme Q is ubiquinone.
(4) The solid of (1) or (2), wherein the coenzyme Q is ubiquinol.
(5) The solid of any of (1) to (4), wherein at least a part of coenzyme Q is amorphous.
(6) The solid of any of (1) to (5), wherein the fine particles of coenzyme Q have an average particle size of not more than 5 μm.
(7) The solid of any of (1) to (6), wherein the physically crosslinked polymer is obtained from a water-soluble polymer having property of forming a physical gel.
(8) The solid of (7), wherein the aforementioned water-soluble polymer is one or more kinds selected from the group consisting of aqueous alginic acid, a derivative thereof, low methoxyl pectin, gelatin, xanthan gum, carmellose sodium, carageenan, aqueous cellulose and a derivative thereof.
(9) The solid of (7), wherein the aforementioned water-soluble polymer is selected from aqueous alginic acid, a derivative thereof and/or gelatin.
(10) The solid of any of (1) to (9), further comprising an emulsifier and/or fats and oils.
(11) A method of producing a solid comprising coenzyme Q, which comprises a step of contacting a first aerosol or droplet liquid comprising a water-soluble polymer solution having property of forming a physical gel and coenzyme Q with a second aerosol liquid comprising a gelling agent.
(12) The method of (11), wherein the solid is a granule.
(13) The production method of (11) or (12), wherein the coenzyme Q is ubiquinone.
(14) The method of (11) or (12), wherein the coenzyme Q is ubiquinol.
(15) The method of any of (11) to (14), wherein the gelling agent is an aqueous calcium chloride solution.
(16) A solid comprising coenzyme Q, which is produced by the method of any of (11) to (15).

### EFFECT OF THE INVENTION

The coenzyme Q-containing solid of the present invention preserves fine particles of coenzyme Q in a dispersion state in the aforementioned physically crosslinked polymer, is quickly disintegrated in the intestine to rapidly release coenzyme Q, and is superior in systemic absorbability. In addition, the production method of the present invention can produce coenzyme Q-containing granules by an industrially advantageous method.

### BRIEF DESCRIPTION OF THE DRAWINGS

FIG. 1 is a longitudinal sectional view of a production apparatus preferably used by the production method of the present invention.

FIG. 2 is an electron micrograph of ubiquinone-containing granules.

FIG. 3 is an enlarged electron micrograph of one particle of ubiquinone-containing granules.

### EXPLANATION OF SYMBOLS

1 coagulation chamber
2 atomization means of ubiquinone-containing water-soluble polymer
3 atomization means of gelling agent
4 falling water supply means
5 gel produced in coagulation chamber

### BEST MODE FOR CARRYING OUT THE INVENTION

The present invention is explained in detail in the following.

The solid of the present invention is characteristically comprised of particles (granules) comprising a physically crosslinked polymer, and fine particles of coenzyme Q dispersed therein. Accordingly, it is a concept including not only the particles themselves but also bigger ones such as a tablet and the like produced from the particles.

Examples of the coenzyme Q include ubiquinone and ubiquinol. Of the ubiquinone, preferred is one having a number of repeat structures in the isoprene side chain of 10 (oxidized coenzyme Q10). In addition, of the ubiquinol, preferred is one having a number of repeat structures in the isoprene side chain of 10 (reduced coenzyme Q10).

The “physically crosslinked polymer” in the present invention is a polymer wherein a crosslinked state is formed by hydrogen binding, ion binding, chelate formation and the like between polymers. In the present invention, the above-mentioned “physically crosslinked polymer” is preferably obtained from a water-soluble polymer having property of forming a physical gel. Here, the “physical gel” means a gel wherein a crosslinked state is formed, and “having property of forming physical gel” means having property allowing visually observed change from a viscous fluid (zol) to an elastic solid (gel) by addition of an inorganic salt or acid, or a gelling operation such as heating or cooling and the like, to an aqueous solution of a water-soluble polymer.

While the water-soluble polymer having property of forming physical gel, to be used in the present invention, is not particularly limited as long as the above-mentioned property can be expressed, for example, aqueous alginic acid and a derivative thereof, low methoxyl pectin, gelatin, xanthan gum, carmellose sodium, carageenan, polyvinylpyrrolidone, aqueous cellulose and a derivative thereof and the like can be mentioned, which may be used alone or in a combination of two or more kinds. Among these, in the present invention, aqueous alginic acid and a derivative thereof, and gelatin can be used preferably. Here, preferred as the derivative is one that can achieve the object of the present invention, and it is a concept including salts.

The aqueous alginic acid and a derivative thereof to be preferably used in the present invention are not limited as long as they have property to form physical gel by reaction with a polyvalent metal salt or acid, for example, alginic acid, sodium alginate, potassium alginate, ammonium alginate and the like. Alginic acid is a long chain copolymer of D-mannuronic acid (M) and L-glucuronic acid (G), where the component ratio of the both, that is, M/G ratio, is known to greatly affect the properties. The alginic acid and a derivative thereof to be used in the present invention are not particularly limited as to their derivation, and one derived from microorganism produced from the genus Azotobacter or the genus Pseudomonas, and an extract from a plant such as seaweed and the like can be used. While the molecular weights of the alginic acid and a derivative thereof to be used in the present invention are not particularly limited, preferred from the aspect of transferability of solution on production are an M/G ratio of 0.1-1.5, and a viscosity of 1 wt % aqueous solution of 10-2000 cps when measured at 25° C.

The gelatin to be used in the present invention is not particularly limited as regards the derivation thereof, kind and the like, and can be appropriately selected according to the use etc. of the solid and the like. For example, one derived from the skin of cow, pig or fish can be used.

In the coenzyme Q containing solid of the present invention, the average particle size of fine particles of coenzyme Q dispersed in the solid is preferably not more than 5 μm, more preferably not more than 1 μm. While the lower limit of the average particle size is not particularly limited, it is generally not less than 0.1 μm.

The average particle size is measured by a dynamic light scattering particle size analyzer.

The production method of the coenzyme Q-containing solid of the present invention is not particularly limited as long as it can afford a solid comprising fine particles of coenzyme Q dispersed in polymer particles. For example, the solid can be obtained by adding coenzyme Q to a solution of a water-soluble polymer and mixing them to give a water-soluble polymer solution containing coenzyme Q, emulsifying the polymer solution as necessary, and gelling the water-soluble polymer by adding a gelling agent or applying an operation such as heating, cooling and the like.

When coenzyme Q is added to a water-soluble polymer solution, coenzyme Q is preferably heated to a temperature higher than the melting point thereof to cause thermal melting, and added to an aqueous polymer solution heated in the same manner and emulsified.

In addition, by adding an emulsifier to the water-soluble polymer solution as necessary, a stabler emulsion can also be formed. The emulsifier to be used is preferably one used for pharmaceutical products and foods, and examples thereof include monoglycerol fatty acid ester, monoglycerol fatty acid organic acid ester, polyglycerin fatty acid ester, polyglycerol-condensed ricinoleic acid ester, sucrose fatty acid ester, propylene glycol fatty acid ester, sorbitan fatty acid ester, sorbitan polyoxyethylene fatty acid ester, lecithin and the like. Particularly, polyglycerin fatty acid ester is preferable.

Where necessary, moreover, fats and oils as an absorption promoter may be added to the water-soluble polymer solution. Fat and oil is not particularly limited and, for example, may be natural fat and oil from plant or animal, or synthetic fat and oil, or processed fat and oil. More preferably, it is fat and oil acceptable for foods, feeds, pharmaceutical agents and the like.

Examples of the vegetable fat and oil include coconut oil, palm oil, palm kernel oil, flaxseed oil, camellia oil, brown rice germ oil, rape seed oil, rice oil, peanuts oil, corn oil, wheat germ oil, soy bean oil, perilla oil, cottonseed oil, sunflower kerel oil, kapok oil, evening primrose oil, shea butter, sal butter, cacao butter, sesame oil, safflower oil, olive oil, pomegranate oil, bitter gourd oil and the like, and examples of animal fats and oils include lard, milk fat, fish oil, beef fat and the like. In addition, they include medium chain triglyceride wherein each fatty acid has a carbon number of 6-12, preferably 8-12, fats and oils obtained by processing them by fractionation, hydrogenation, transesterification etc. and partial glycerides thereof.

The ratio of coenzyme Q and a water-soluble polymer solution in the present invention varies depending on the desired properties of a solid and cannot be defined uniformly. For example, 0.01-70 parts by weight, preferably 0.1-50 parts by weight, more preferably 1-10 parts by weight of coenzyme Q is added to 100 parts by weight of a water-soluble polymer solution such as aqueous alginic acid, a derivative thereof and the like, and the above-mentioned emulsifier and fats and oils are added as necessary to form an O/W emulsion. An emulsion can be formed by using a homomixer, a homogenizer, a high-pressure homogenizer, a polytron and the like.

In the thus-obtained emulsion, the average particle size of the emulsion particles is desirably not more than 5 μm, more preferably not more than 1 μm, generally not less than 0.1 μm, for the stability of emulsion and smooth absorption of coenzyme Q and the object solid after ingestion. The average particle size of emulsion particles is a median particle size (50% particle size), which can be measured, for example, by a dynamic light scattering particle size analyzer (LB-550 manufactured by Horiba, Ltd.). Since the size of the emulsion particles becomes almost the same as that of ubiquinone fine particles dispersed in the obtained solid, the particle size of coenzyme Q fine particles dispersed in the obtained solid can be controlled by adjusting the average particle size of the emulsion particles here.

Then, the water-soluble polymer can be gelled by adding a gelling agent or applying an operation such as heating, cooling and the like. The gelling agent (coagulating agent) usable in the present invention may be any substance having property of gelling the aforementioned water-soluble polymer. When aqueous alginic acid or a derivative thereof, carageenan or a derivative thereof, or low methoxyl pectin is selected as the water-soluble polymer, since a physical gel is formed by using the aqueous polyvalent metal salt solution, an aqueous solution of calcium chloride, magnesium chloride, or barium chloride is preferably used as a gelling agent. The water-soluble polymer solution may be used alone or in a combination of two or more kinds thereof, according to which one or more kinds of the corresponding gelling agents are used. When gelatin alone is used as a water-soluble polymer, and the like, a gelling agent may not necessarily be required.

While the amount of the gelling agent (coagulating agent) to be used is not necessarily limited, it is preferably 0.2-30 parts by weight, more preferably 0.5-15 parts by weight, relative to 100 parts by weight of a water-soluble polymer. This is because when the amount of the gelling agent (coagulating agent) to be used is less than 0.2 part by weight, coagulation of the water-soluble polymer sometimes becomes insufficient, and when the amount of the gelling agent (coagulating agent) to be used exceeds 20 parts by weight, the amount of the gelling agent (coagulating agent) in the drainage water increases, and the load on the drainage treatment tends to increase, though the coagulation property is not influenced.

As a method of contacting a coenzyme Q-containing water-soluble polymer solution with a coagulating agent (gelling agent) in the present invention, for example, a coenzyme Q-containing water-soluble polymer solution preferably in an emulsion state is continuously atomized or added dropwise into a coagulative gaseous phase atmosphere where a predetermined amount of an aqueous solution of a coagulating agent (gelling agent) is atomized continuously in an aerosol state to allow contact, though the method is not limited to the above.

The aerosol state is not particularly limited as long as it is what is called a mist state. The volume average droplet diameter of a gelling agent droplet is preferably 0.01-10 μm. As the atomization means, a high-pressure nozzle, a Pneumatic spray nozzle, an ultrasonication nozzle, a high frequency nozzle, a rotating disk and the like are used.

In the present invention, since coagulation can proceed in a gaseous phase while retaining the droplet shape of a coenzyme Q-containing water-soluble polymer solution atomized or added dropwise into a gaseous phase. Therefore, the droplet diameter of the coenzyme Q-containing water-soluble polymer solution during atomization or dropwise addition can be adjusted freely in accordance with the supply form of a dry granule product. The volume average droplet diameter is generally within the range of 10 μm-1000 μm, preferably 50 μm-200 μm. The droplet diameter during atomization or dropwise addition of a coenzyme Q-containing water-soluble polymer solution can be indirectly determined by the measurement of particle size distribution of the volume average particle size of the produced gel.

In the present invention, a coenzyme Q-containing water-soluble polymer solution atomized in a gaseous phase is brought into contact with a gelling agent (coagulating agent) capable of coagulating the coenzyme Q-containing water-soluble polymer solution to promote coagulation.

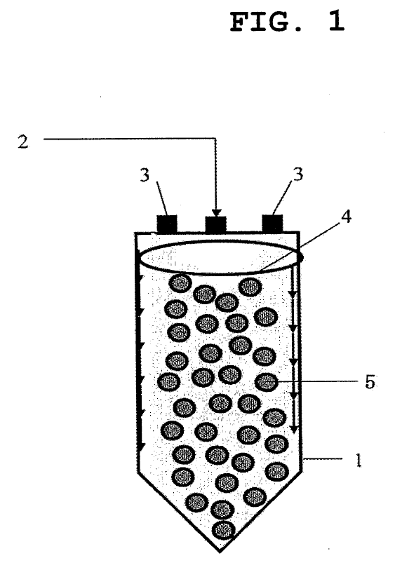

A schematic longitudinal sectional view of a production apparatus preferably used in the above-mentioned production method is shown in FIG. 1, and explained in the following. In the Figure, 1 is a coagulation chamber, and the shape of the coagulation chamber is not particularly limited. Generally, a cylindrical chamber with a hollow conical bottom is used.

In the present invention, to prevent deformation of a coenzyme Q-containing water-soluble polymer solution due to the impact of entry into the liquid phase from the gaseous phase, gelling is desired to be completed in the gaseous phase, and a certain height is required from the liquid surface of the aqueous phase to the position of atomization or dropwise addition. The minimum height from the liquid surface of the aqueous phase to the position of atomization or dropwise addition mentioned above is preferably not less than 1.0 m, more preferably not less than 1.5 m. While the maximum height from the liquid surface of the aqueous phase to the position of atomization or dropwise addition is not particularly limited, it is preferably not more than 20 m, more preferably not more than 5.5 m, from the aspect of facility cost. The top of the coagulation chamber is provided with an atomization means 2 and a gelling agent atomization means 3 for dispersing the aforementioned coenzyme Q-containing water-soluble polymer solution as a droplet. In addition, a falling water supply means 4 is provided to supply water on the wall to avoid attachment of gel produced on the wall of the coagulation chamber. To be specific, a cylindrical pipe having many pores facing the side wall is used to continuously supply water. Gel 5 produced in the coagulation chamber falls due to the gravity and becomes particles, which are recovered as water suspension.

The coenzyme Q produced by the above-mentioned method includes non-crystalline form (amorphous). Noncrystalline coenzyme Q is contained in a proportion of generally not less than 50%, preferably not less than 60%, more preferably not less than 80%. By the presence of noncrystalline coenzyme Q, absorbability in the living body is expected to be preferable.

Thereafter, the coenzyme Q-containing granule of the present invention can be obtained by dehydration and drying according to a conventional method.

The solid of the present invention obtained as mentioned above is generally in the form of granules. The average particle size of the granules is 10 μm-1000 μm, preferably 20 μm-500 μm, more preferably 20 μm-200 μm, and they may be taken as granules, or may be used by mixing in a food and the like. Where necessary, the granules can be filled in a capsule and utilized as a capsule agent, or compression molded together with an excipient by a conventional means and utilized as one embodiment of a solid, i.e., tablet. In the present invention, the solid is a concept encompassing the granule itself, and a solid agent derived therefrom such as tablet and the like. Here, the solid agent is a concept encompassing not only pharmaceutical agents but also foods (functional food, health food, supplement etc.) and the like. The average particle size of the granule is measured by a laser diffraction particle size analyzer and the like.

In the case of a capsule, it is produced by filling ubiquinone containing granules as mentioned above in a capsule shell made of gelatin etc. by a known method. In the case of a tablet, it is produced by compression molding the above-mentioned granule and excipients such as lactose, mannitol, crystalline cellulose, starch and the like, or the mixture of the granules and excipients added with, where necessary, disintegrants such as hydroxypropyl cellulose, carboxymethyl cellulose and the like, and lubricants such as talc, magnesium stearate and the like, using a tableting machine and the like according to a known method.

### EXAMPLES

The present invention is explained in more detail in the following by referring to Examples, which are not to be construed as limitative.

### Preparation of Coenzyme Q10-Containing Emulsion

### Preparation Example 1

“KANEKA Q10” (manufactured by Kaneka, 20 g) was melted by heating to 60° C., and the solution was dispersed in an aqueous solution (1 L) and prepared to 60° C. in advance containing sodium alginate (IL6-G manufactured by KIMIKA, 20 g). The mixture was emulsified using a homogenizer under the conditions of 15000 rpm, 10 min. The particle size distribution of the coenzyme Q10-containing emulsion particles in the uniform emulsion was measured by a dynamic light scattering particle size analyzer (LB-550 manufactured by Horiba, Ltd.). As a result, the median particle size was 3.30 μm.

### Preparation Example 2

“KANEKA Q10” (manufactured by Kaneka, 20 g) was melted by heating to 60° C., and the solution was dispersed in an aqueous solution (1 L) and prepared to 60° C. in advance containing sodium alginate (IL6-G manufactured by KIMIKA, 20 g) and gelatin (nitta-gelatin APH, 50 g). The mixture was emulsified using a homogenizer under the conditions of 15000 rpm, 10 min. The particle size distribution of the coenzyme Q10-containing emulsion particles in the uniform emulsion was measured by a dynamic light scattering particle size analyzer (LB-550 manufactured by Horiba, Ltd.). As a result, the median particle size was 0.79 μm.

### Preparation Example 3

In the same manner as in Preparation Example 1 except that decaglycerol monooleic acid ester (J-0381V manufactured by RIKEN Vitamin Co. Ltd., 20 g) and medium-chain triglyceride (Actor M-2 manufactured by RIKEN Vitamin Co. Ltd., 10 g) were added to the composition of Preparation Example 1, an emulsion liquid was prepared.

The particle size distribution of the emulsion particles was measured by a dynamic light scattering particle size analyzer (LB-550 manufactured by Horiba, Ltd.). As a result, the median particle size was 0.20 μm.

### Example 1

### Preparation of Coenzyme Q10-Containing Granule

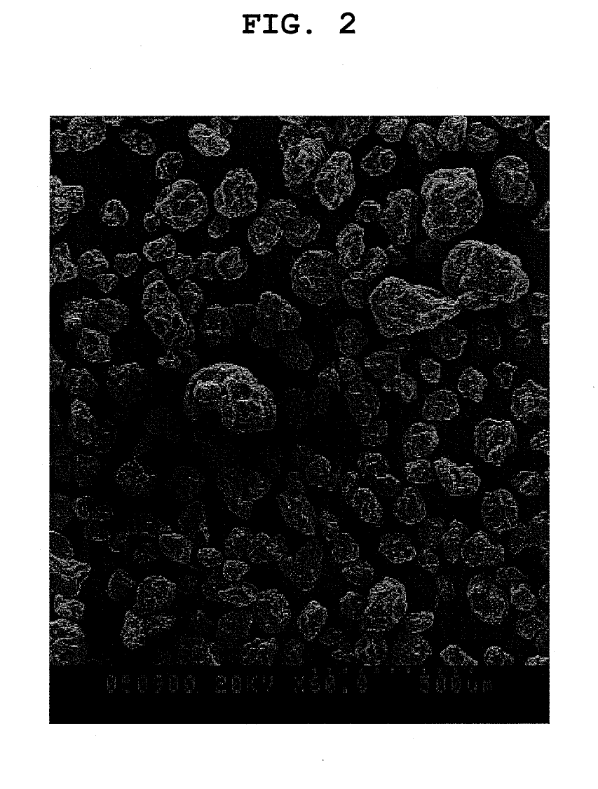

The coenzyme Q10-containing emulsion liquids obtained in Preparation Examples 1 to 3 were atomized from the top of a cylindrical coagulation chamber (inner diameter 45 cm, total height about 5 m) using a pneumatic spray nozzle (BIMJ2004 manufactured by H. IKEUCHI & Co., Ltd.) as an atomization means under the conditions of volume average droplet diameter 150 μm, supply amount 150 g/min. Simultaneously, 30 wt % aqueous calcium chloride solution was atomized at a volume average droplet diameter of 0.1-10 μm using a pneumatic spray nozzle (1/4J series SU13A manufactured by Spraying Systems Co., Japan) while mixing with air such that the calcium chloride solid content would be 5-15 parts by weight relative to 100 parts by weight of the emulsion liquid. To prevent the coenzyme Q10 emulsion liquid atomized from the top of the coagulation chamber from attaching to the wall surface of the coagulation chamber, moreover, distilled water at 25° C. was continuously supplied at 6 L/min using a pipe having an inner diameter of about 20 mm with many 2 mmφ pores facing the side wall. The coenzyme Q10-containing emulsion that dropped through the coagulation chamber and became gel in a particle state was recovered from the bottom of the coagulation chamber as a water suspension. The recovered suspension was dehydrated and dried by conventional methods to give granules. It was confirmed with an electron microscope that granules having a volume average particle size of about 50 μm were produced from all of the emulsion liquids of Preparation Examples 1 to 3. FIG. 2 shows an electron micrograph of the granules obtained by forming a gel having the composition of Preparation Example 2 and drying the gel.

### Example 2

### Confirmation of Dispersion State of Coenzyme Q10-Containing Granules

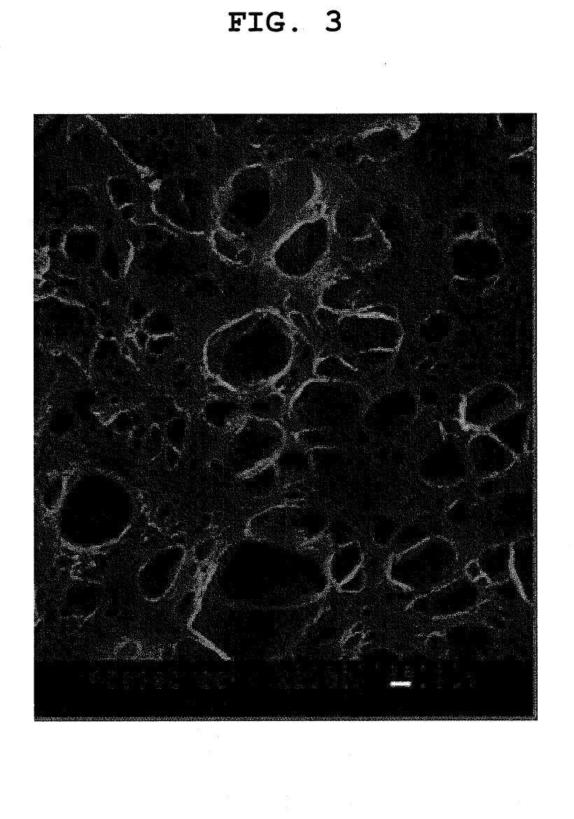

Granules obtained in Example 1 were processed to have a section according to a conventional method, and coenzyme Q10 on the section was dissolved by immersion in hexane for 2 min, which was confirmed with an electron microscope. The sectional view of the granules obtained from the emulsion liquid of Preparation Example 2 is shown in FIG. 3. According to FIG. 3, a 1-2 μm lattice structure was formed and the presence of coenzyme Q10 within the lattice is assumed. Since the size of the lattice and the median particle size of the coenzyme Q10-containing emulsion liquid of Preparation Example 2 are almost the same, it was confirmed that coenzyme Q10 was dispersed while maintaining the emulsion particle size.

### Example 3

### In Vitro Disintegration Test

900 ml each of the 1st fluid and the 2nd fluid defined in the Japanese Pharmacopoeia was prepared, 100 mg (dry weight) of the granules containing coenzyme Q10 obtained in Example 1 was added, and paddling was performed at 37° C., 100 rpm. The solution was sampled at 0, 10, 30, 60, 180 min, and the time-course changes in the particle size distribution were observed by a laser diffraction particle size analyzer (Horiba, Ltd. LA-920), whereby disintegratability of the granules was confirmed. None of the granules obtained from the emulsion liquids of Preparation Examples 1-3 were disintegrated by the 1st fluid, and the particle size thereof was maintained. On the other hand, it was confirmed that the particles were disintegrated in 10 minutes by the 2nd fluid.

TABLE 1

| | 1st fluid | 2nd fluid |
|---|---|---|
| | (artificial gastric | (artificial bowel |
| | fluid) | fluid) |
| Preparation Example | not disintegrated | disintegrated in |
| 1 | even in 180 min | 10 min |
| Preparation Example | not disintegrated | disintegrated in |
| 2 | even in 180 min | 10 min |
| Preparation Example | not disintegrated | disintegrated in |
| 3 | even in 180 min | 10 min |

### Preparation Example 4

In the same manner as in Preparation Example 1 except that “KANEKA QH (registered trade mark)” (manufactured by Kaneka Corporation), which is reduced coenzyme Q10, was used instead of “KANEKA Q10”, an emulsion liquid was obtained.

### Preparation Example 5

In the same manner as in Preparation Example 2 except that “KANEKA QH (registered trade mark)” (manufactured by Kaneka Corporation), which is reduced coenzyme Q10, was used instead of “KANEKA Q10”, emulsification was performed. The particle size distribution of the reduced coenzyme Q10-containing emulsion particles in the uniform emulsion was measured by a dynamic light scattering particle size analyzer (LB-550 manufactured by Horiba, Ltd.). As a result, the median particle size was 1 μm.

### Preparation Example 6

In the same manner as in Preparation Example 3 except that “KANEKA QH (registered trade mark)” (manufactured by Kaneka Corporation), which is reduced coenzyme Q10, was used instead of “KANEKA Q10”, an emulsion liquid was obtained.

### Example 4

### Preparation of Reduced Coenzyme Q10-Containing Granule

The reduced coenzyme Q10-containing emulsion liquids obtained in Preparation Examples 4 to 6 were atomized from the top of a cylindrical coagulation chamber (inner diameter 45 cm, total height about 5 m) using a pneumatic spray nozzle (BIMJ2004 manufactured by H. IKEUCHI & Co., Ltd.) as an atomization means under the conditions of volume average droplet diameter 150 μm, supply amount 150 g/min. Simultaneously, 30 wt % aqueous calcium chloride solution was atomized at a volume average droplet diameter of 0.1-10 μm using a pneumatic spray nozzle (1/4J series SU13A manufactured by Spraying Systems Co., Japan) while mixing with air such that the calcium chloride solid content would be 5-15 parts by weight relative to 100 parts by weight of the emulsion liquid. To prevent the reduced coenzyme Q10 emulsion liquid atomized from the top of the coagulation chamber from attaching to the wall surface of the coagulation chamber, moreover, distilled water at 25° C. was continuously supplied at 6 L/min using a pipe having an inner diameter of about 20 mm with many 2 mmφ pores facing the side wall. The reduced coenzyme Q10-containing emulsion that dropped through the coagulation chamber and became gel in a particle state was recovered from the bottom as a water suspension. The recovered suspension was dewatered and dried by conventional methods to give granules. It was confirmed with an electron microscope that granules having a volume average particle size of about 50 μm were produced from all of the emulsion liquids of Preparation Examples 4 to 6.

### Example 5

### Measurement of Crystallization Rate of Coenzyme Q10 in Granule

Calorimetric analysis of coenzyme Q10 granules obtained in Example 1 and Example 4 and oxidized coenzyme Q10 powder (Kaneka-coenzyme Q10) used as the starting material in Preparation Examples 1-3 by a differential scanning calorimetric analyzer (manufactured by SII; EXSTAR6000 DSC6220) was performed under the following conditions. The results are shown in Table 2. The crystallization rate was calculated from the measurement value of the melting heat (ΔH).

Analysis conditions; 20° C.→80° C. (5° C./min)→−50° C. (−5° C./min)

TABLE 2

| | crystallinity |
|---|---|
| sample | degree |
| Example 1 (granule obtained from emulsion | 11% |
| liquid of Preparation Example 2) | |
| Example 4 (granule obtained from emulsion | 34% |
| liquid of Preparation Example 4) | |
| Example 4 (granule obtained from emulsion | 34% |
| liquid of Preparation Example 5) | |
| Example 4 (granule obtained from emulsion | 36% |
| liquid of Preparation Example 6) | |
| Kaneka coenzyme Q10 | 100% |

As a result, it was confirmed that coenzyme Q10 granule of the present invention contains amorphous coenzyme Q10, though generally commercially available coenzyme Q10 powder is 100% crystal.

While some of the embodiments of the present invention have been described in detail in the above, those of ordinary skill in the art can make various modifications and changes to the particular embodiments shown without substantially departing from the teaching and advantages of the present invention. Such modifications and changes are encompassed in the spirit and scope of the present invention as set forth in the appended claims.

This application is based on a patent application No. 2006-119409 filed in Japan, the contents of which are incorporated in full herein by this reference.

1. A solid comprising coenzyme Q, wherein fine particles of coenzyme Q are dispersed in particles comprised of physically crosslinked polymers.

2. The solid of claim 1, wherein the solid is a granule.

3. The solid of claim 1, wherein the coenzyme Q is ubiquinone.

4. The solid of claim 1, wherein the coenzyme Q is ubiquinol.

5. The solid of claim 1, wherein at least a part of coenzyme Q is amorphous.

6. The solid of claim 1, wherein the fine particles of coenzyme Q have an average particle size of not more than 5 μm.

7. The solid of claim 1, wherein the physically crosslinked polymer is obtained from a water-soluble polymer having property of forming a physical gel.

8. The solid of claim 7, wherein said water-soluble polymer is one or more kinds selected from the group consisting of aqueous alginic acid, a derivative thereof, low methoxyl pectin, gelatin, xanthan gum, carmellose sodium, carageenan, aqueous cellulose and a derivative thereof.

9. The solid of claim 7, wherein said water-soluble polymer is selected from aqueous alginic acid, a derivative thereof and/or gelatin.

10. The solid of claim 1, further comprising an emulsifier and/or fats and oils.

11. A method of producing a solid comprising coenzyme Q, which comprises a step of contacting a first aerosol or droplet liquid comprising a water-soluble polymer solution having property of forming a physical gel and coenzyme Q with a second aerosol liquid comprising a gelling agent.

12. The method of claim 11 wherein the solid is a granule.

13. The method of claim 11, wherein the coenzyme Q is ubiquinone.

14. The method of claim 11, wherein the coenzyme Q is ubiquinol.

15. The method of claim 11, wherein the gelling agent is an aqueous calcium chloride solution.

16. (canceled)
